(19) United States
(12) Patent Application Publication
(10) Pub. No.: US 2026/0109797 A1
(43) Pub. Date: Apr. 23, 2026

(54) PRODUCTION METHOD FOR DEGRADING COCOA SHELL INTO POLYSACCHARIDE FOR GRAFTING ONTO POLYMER AND YARN THEREOF

(71) Applicant: Si-Rong CHANG, New Taipei City (TW)

(72) Inventor: Si-Rong CHANG, New Taipei City (TW)

(21) Appl. No.: 18/922,412

(22) Filed: Oct. 22, 2024

(51) Int. Cl. C08F 251/00; C08F 4/34; C08F 6/10; C12P 19/04

(52) U.S. Cl. CPC C08F 251/00; C08F 4/34; C08F 6/10; C12P 19/04; C08F 2400/00

(57) ABSTRACT

A production method for degrading cocoa shell into polysaccharide for grafting onto polymer includes: preparing anaerobic thermophilic lignin-decomposing bacteria and cocoa shell powder; adding the cocoa shell powder into a decomposing bacteria culture solution with anaerobic thermophilic lignin-decomposing bacteria to obtain a cocoa shell degradation aqueous solution; obtaining a degraded saccharide liquid from the cocoa shell degradation aqueous solution to undergo high temperature melting with sorbitol and citric acid so as to polymerize into a polysaccharide powder; then placing the polysaccharide powder, N-isopropylacrylamide (NIPAAm), and an initiator into a reaction vessel filled with ethanol to obtain a polysaccharide-grafted N-isopropylacrylamide (NIPAAm) powder; and finally, performing a blending process with a polymer to complete covalent bonding of the polysaccharide-grafted N-isopropylacrylamide (NIPAAm) powder and the polymer. In addition, a plastic masterbatch can then be made for drawing into yarns and weaving into fabrics.

### BACKGROUND OF THE INVENTION

### (a) Technical Field of the Invention

The present invention relates to a production method for degrading cocoa shell into polysaccharide for grafting onto polymer and yarn thereof, and more particularly to degrading cocoa shells, as an agricultural waste derived from chocolate, into monosaccharide or polysaccharide, which are then polymerized into polysaccharide to be covalently bonded to polymer through grafting technology for being subsequently made into textile yarns to thereby significantly increase the reuse rate of agricultural waste from cocoa shells.

### (b) Description of the Prior Art

Cocoa is a small tropical evergreen tree, which is mainly cultivated in the tropical areas around the Tropic of Cancer and the Tropic of Capricorn. The pulp and seeds (cocoa beans) are extracted from the cocoa after processing. The remaining shell accounts for 80% of the whole fruit, and this means a large amount of waste needs to be processed. In addition to causing thorny and difficult problems for agricultural enterprises, the processing of this waste will also increase the cost burden of agricultural enterprises.

The current methods of burying or burning husk or shell agricultural waste will cause pollution to land and air, and increase the burden of cleanup costs for agricultural enterprises. Therefore, adopting a method to achieve value-added applications and related product development technology for cocoa shells is the best option to deal with cocoa shell waste today.

Evaluating the cocoa shell from the point of view of the textile technology would be such that as the man-made polyester fiber is made of plastics, it and the plastic waste generated from daily consumption are difficult to decompose, and eventually become more serious plastic waste. However, these plastic wastes flow with rainwater and rivers to eventually flow from the land into the sea. Whether in the form of plastic blocks or microplastics, the pollution to the marine ecology and environment is becoming increasingly serious.

Therefore, it is the main subject that the present invention aims to resolve the problem by developing a method that the waste cocoa shells after extracting the cocoa pulp and seeds may be subjected to biodegradation to decompose lignocellulose, followed by degradation and then polymerization into polysaccharide to be then covalently bonded with polymer to make textile yarns, so as to not only greatly improve the reuse rate of cocoa shell agricultural waste, but also to indeed achieve a degradation effect when soaked in seawater, making it marine organism decomposable plastics, thus avoiding pollution of the marine ecological environment.

### SUMMARY OF THE INVENTION

The primary objective of the present invention is to provide a production method for degrading cocoa shell into polysaccharide for grafting onto polymer, and the production method comprises:
Step 1, preparing anaerobic thermophilic lignin-decomposing bacteria;
Step 2, preparing a cocoa shell powder of cocoa shell;
Step 3, preparing a cocoa shell degradation aqueous solution: adding the cocoa shell powder into a decomposing bacteria culture solution with the anaerobic thermophilic lignin-decomposing bacteria, wherein during a course of preparation, the pH value of the decomposing bacteria culture solution is set to be between 4-8 and after a preset time, the anaerobic thermophilic lignin-decomposing bacteria degrade the cocoa shell powder to obtain a cocoa shell degradation aqueous solution; and
Step 4, polymerizing the cocoa shell degradation aqueous solution into polysaccharide and performing grafting processing: subjecting the cocoa shell degradation aqueous solution to a centrifugation process to obtain a degraded saccharide liquid and setting a saccharide content of the saccharide liquid to be between 70%-80%, and adding sorbitol and citric acid to carry out high temperature melting for polymerization to obtain a polysaccharide powder; placing the polysaccharide powder and N-isopropylacrylamide (NIPAAm) in a reaction vessel filled with ethanol and added with an initiator (such as dibenzoyl peroxide) to carry out a preset reaction process and carrying out a drying process after the reaction process is completed to obtain a polysaccharide-grafted N-isopropylacrylamide (NIPAAm) powder; and finally, subjecting the polysaccharide-grafted N-isopropylacrylamide (NIPAAm) powder to a blending process with a polymer to allow the polysaccharide-grafted N-isopropylacrylamide (NIPAAm) powder to covalently bond to the polymer to complete a polysaccharide-grafted N-isopropylacrylamide (NIPAAm) blended polymer.

As such, the above-described production method for degrading cocoa shell into polysaccharide for grafting onto polymer is that cocoa shells, which are agricultural waste derived from a chocolate manufacturing process, are reused, mainly using anaerobic thermophilic lignin decomposing bacteria to have the cocoa shell degraded to form monosaccharides or polysaccharides, and then sorbitol and citric acid are added for high-temperature melting to polymerize into polysaccharide, and finally, N-isopropylacrylamide (NIPAAm) is added and covalently bonded, through grafting technology, to polymer to complete the production method for degrading cocoa shell into polysaccharide for grafting onto polymer according to the present invention. As such, a plastic masterbatch including a polysaccharide-grafted N-isopropylacrylamide (NIPAAm) blended polymer (such as polypropylene (PP)) is produced to thereby achieve greatly increasing the reuse rate of cocoa shell agricultural waste, and also, the polymer or masterbatch thereof produced according to the present invention can be soaked in sea water to indeed fulfill an effect of degradation, and avoiding polluting the marine ecology and environment.

A secondary objective of the present invention is to provide a production method for yarn of cocoa shell degraded into polysaccharide grafted onto polymer, and the production method comprises: Steps 1-4 of the above-described production method for degrading cocoa shell into polysaccharide for grafting onto polymer, followed by Step 5, a yarn drawing process: subjecting a plastic masterbatch of polysaccharide-grafted N-isopropylacrylamide (NIPAAm) blended polymer (such as polypropylene (PP), polyester (PET), and nylon 6 (PA6)) to a yarn drawing operation to make a textile yarn of the polysaccharide grafted N-isopropylacrylamide (NIPAAm) blended polymer, which can be then woven into fabric to achieve significantly increasing the reuse rate of cocoa shell agricultural waste, and further, the textile yarn or fabric made according to the present invention can be soaked in seawater to indeed fulfill an effect of degradation, and avoiding polluting the marine ecology and environment.

### BRIEF DESCRIPTION OF THE DRAWINGS

FIG. 1 illustrates a polysaccharide molecular formula of polysaccharide degraded from cocoa shell according to the present invention.

FIG. 2 illustrates a molecular formula of N-isopropylacrylamide for grafting provided according to the present invention.

FIG. 3 illustrates a molecular formula of dibenzoyl peroxide as an organic compound provided according to the present invention.

FIG. 4 illustrates a molecular formula of N-isopropylacrylamide and polysaccharide provided according to the present invention.

FIG. 5 is a schematic view illustrating a molecular formula of N-isopropylacrylamide grafted with polysaccharide provided according to the present invention.

FIG. 6 is a schematic view illustrating a grafted structure of covalently bonded N-isopropylacrylamide powder and polypropylene (PP) powder provided according to the present invention.

FIG. 7 illustrates a test report for testing of feasibility of fabric degradation by marine seawater (marine-degradability ASTM D6691) for polysaccharide blended polyester PET fabric according to the present invention.

FIG. 8 illustrates a test report for testing of feasibility of fabric degradation by marine seawater (marine-degradability ASTM D6691) for polysaccharide blended nylon PA6 fabric according to the present invention.

FIG. 9 is a flow chart illustrating a production method for degrading cocoa shell into polysaccharide for grafting onto polymer provided according to the present invention.

FIG. 10 is a flow chart illustrating a production method for yarn of cocoa shell degraded into polysaccharide grafted onto polymer provided according to the present invention.

### DETAILED DESCRIPTION OF THE PREFERRED EMBODIMENTS

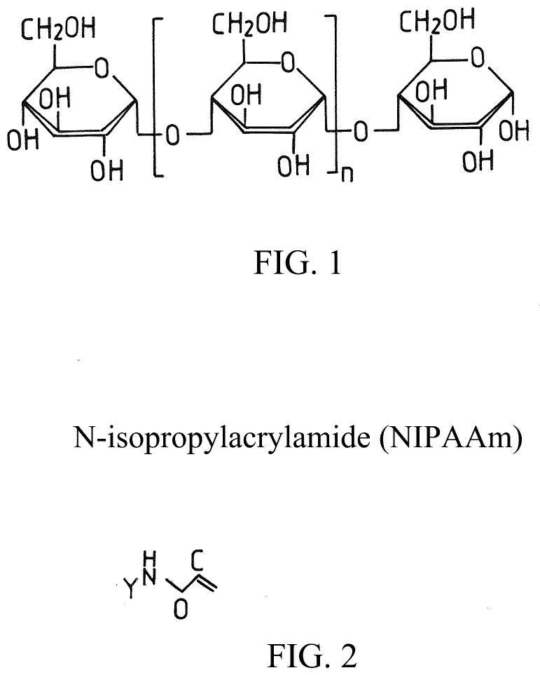

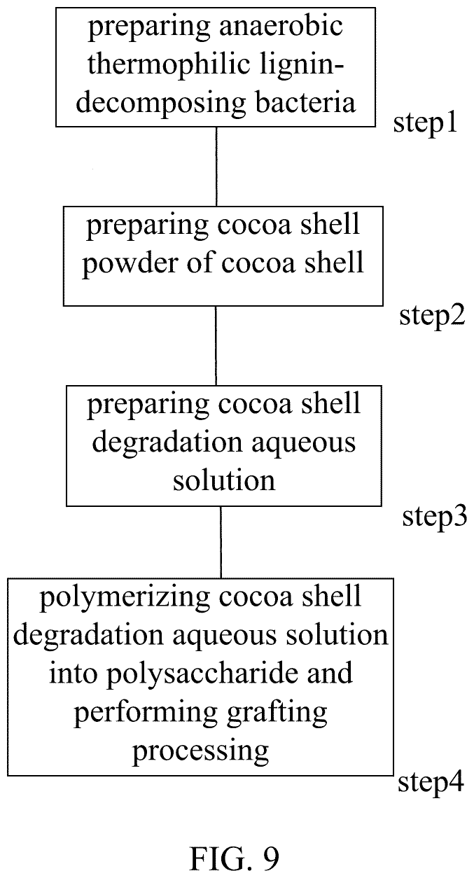

Refer jointly to FIGS. 1-10, the present invention discloses a production method for degrading cocoa shell into polysaccharide for grafting onto polymer and yarns thereof. Firstly, as shown in FIG. 9, the production method for degrading cocoa shell into polysaccharide for grafting onto polymer comprises the following steps:
Step 1: Preparing anaerobic thermophilic lignin-decomposing bacteria; selecting anaerobic thermophilic lignin-decomposing bacteria to be cultured anaerobically. For example, during the anaerobic culture process, sodium sulfide (Na₂S) is added as a reducing agent to maintain the anaerobic environment in the culture medium; in addition, the culture medium prepared under normal conditions can be heated and boiled for 5 to 15 minutes, and is exposed to nitrogen (N₂) using a gas station to remove dissolved oxygen in the culture medium, and then the culture medium is supplied with a mixture of nitrogen (N₂) and carbon dioxide (CO₂), and then the anaerobic thermophilic lignin-decomposing bacteria are subjected to exposure through pumping and evacuating nitrogen for several times to be then added into the culture medium to maintain the best anaerobic state of anaerobic culture. During the anaerobic culture process, glucose is used as a carbon source, and the temperature is set to about 40-70 degrees Celsius, and the pH value is between 5-8.
Step 2: Preparing a cocoa shell powder of cocoa shell; firstly, reducing moisture content of the cocoa shell to less than 15%, and then subjecting the cocoa shell to drying, wherein during the drying process, the temperature is set to 120 degrees Celsius and the drying time is set for 10-24 hours, so that the moisture content of the cocoa shell is below 0.5%; then, subjecting the cocoa shell to crushing in a staged and sectioned manner to finer than 8000 mesh to obtain the cocoa shell powder, thereby making a particle size of the cocoa shell powder about 1 micron or finer.
Step 3: Preparing a cocoa shell degradation aqueous solution; adding the cocoa shell powder into a decomposing bacteria culture solution with the anaerobic thermophilic lignin-decomposing bacteria, wherein during the course, the pH value of the decomposing bacteria culture solution is set to be between 4-8 and after a preset time (such as 5-days), the anaerobic thermophilic lignin-decomposing bacteria degrade the cocoa shell powder to obtain a cocoa shell degradation aqueous solution.

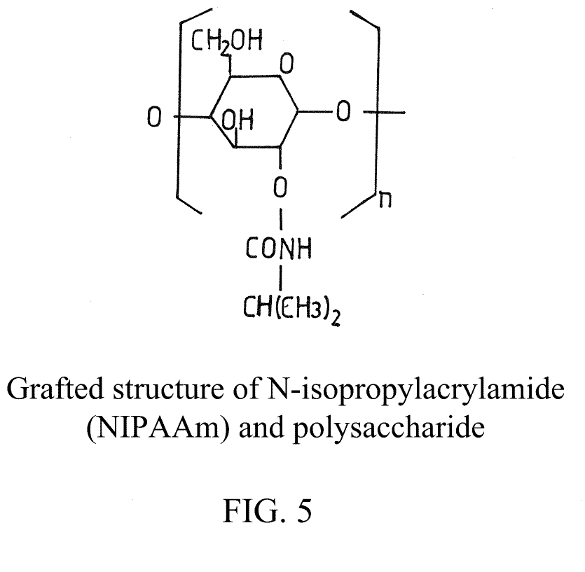

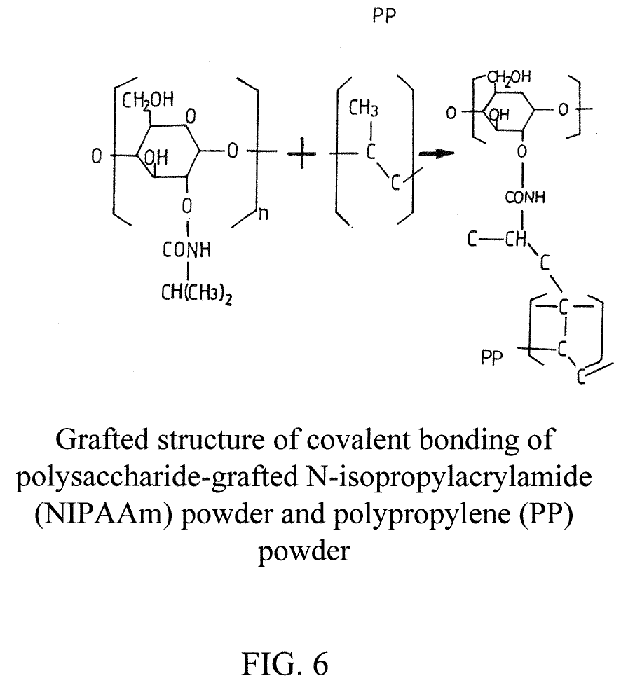

In Step 3, the weight ratio of the cocoa shell powder and decomposing bacteria culture solution is between 1:5-1:10.
Step 4: Polymerizing the cocoa shell degradation aqueous solution into polysaccharide and performing grafting processing; subjecting the cocoa shell degradation aqueous solution to a centrifugation process to obtain a degraded saccharide liquid, and controlling and setting a saccharide content of the saccharide liquid to be between 70%-80%, then adding 5%-20% sorbitol and 0.5%-10% citric acid for high temperature melting to polymerize to obtain a polysaccharide powder, wherein the polysaccharide molecular formula of the polysaccharide powder is shown in FIG. 1; placing the polysaccharide powder and N-isopropylacrylamide (NIPAAm) in a reaction vessel filled with ethanol, wherein N-isopropylacrylamide (NIPAAm) is used as a grafting agent, and a schematic molecular structure illustrating joining of N-isopropylacrylamide (NIPAAm) and polysaccharide is shown in FIG. 4 and the molecular formula of N-isopropylacrylamide (NIPAAm) is as shown in FIG. 2, then adding an initiator to carry out a preset reaction process, and then, performing a drying process after the reaction process is completed in order to obtain a polysaccharide-grafted N-isopropylacrylamide (NIPAAm) powder, wherein the structure of the polysaccharide-grafted N-isopropylacrylamide (NIPAAm) is shown in FIG. 5; finally, subjecting the polysaccharide-grafted N-isopropylacrylamide (NIPAAm) powder to a blending process with a polymer to allow the polysaccharide-grafted N-isopropylacrylamide (NIPAAm) powder to covalently bond to the polymer to complete a polysaccharide-grafted N-isopropylacrylamide (NIPAAm) blended polymer, as shown in FIG. 6, in which a schematic grafted structure of polysaccharide-grafted N-isopropylacrylamide (NIPAAm) powder covalently bonded with a polymer (such as polypropylene (PP)) powder is shown.

In Step 4, in the centrifugation process, the cocoa shell degradation aqueous solution is centrifuged at a high speed of 8000-10000RPM to obtain the degraded saccharide liquid.

In Step 4, the conditions set for the high-temperature melting are that a set temperature is between 110-130 degrees Celsius, and the pressure is between 0.01-0.1 atm, such that the polysaccharide powder is obtained through polymerization under these conditions.

In Step 4, the concentration of N-isopropylacrylamide (NIPAAm) as the grafting agent in ethanol is 0.1-0.8 M.

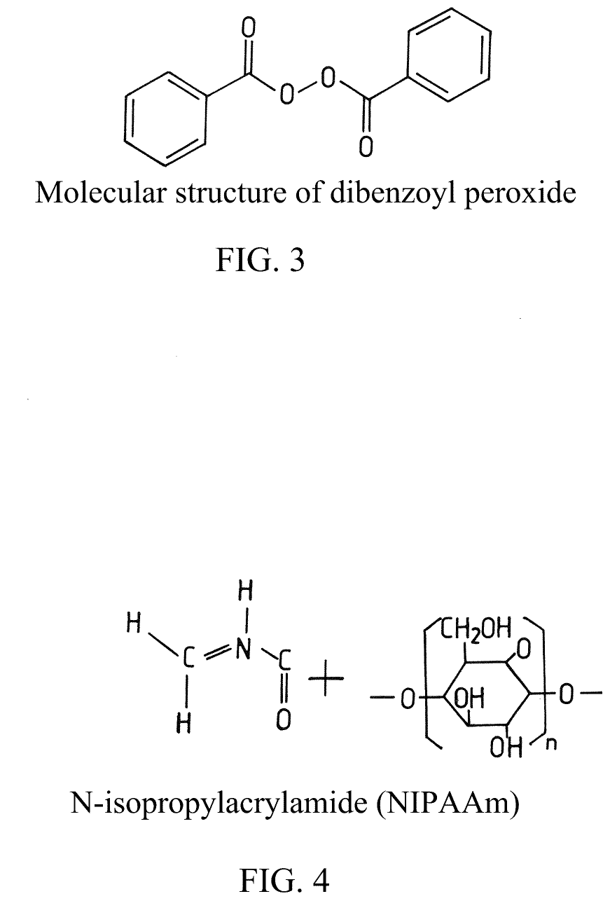

In Step 4, the initiator is dibenzoyl peroxide, which is also known as benzoyl peroxide, commonly known BPO, which is an organic compound with a molecular structural formula shown in FIG. 3; and the concentration of the initiator in ethanol is 1−8×10⁻³ M.

In Step 4, during the reaction process, nitrogen gas is continuously introduced, the reaction time is set to 1-8 hours, and the reaction temperature is controlled to be between 70-90 degrees Celsius.

In Step 4, the polymer can be a plastic material, such as PET (polyethylene terephthalate), PA6 (polyamide (NYLON)), PP (polypropylene), PE (polyethylene), ABS (acrylonitrile-butadiene-styrene copolymer), PC (polycarbonate), PVDF (polyvinylidene fluoride), PS (polystyrene), PES (polyether styrene), PVC (polyvinyl chloride), PAN (polyacrylonitrile), PA6 (nylon 6) and other plastic polymers.

In Sep 4, the blending process is performed with preset plastic masterbatch manufacturing equipment to blend, graft and bond the polysaccharide grafted N-isopropylacrylamide (NIPAAm) powder and the polymer to fulfill the production method for degrading cocoa shell into polysaccharide for grafting onto polymer according to the present invention, so as to finally make a plastic masterbatch of a polysaccharide-grafted N-isopropylacrylamide (NIPAAm) blended polymer. The plastic masterbatch manufacturing equipment has a twin-screw air evacuation mechanism, and the interior of the twin-screw air evacuation mechanism can be in a vacuum state. As such, for example, polysaccharide grafted N-isopropylacrylamide (NIPAAm) powder and the polypropylene (PP) powder as the polymer are first introduced into the twin-screw air evacuation mechanism and stirred at a high speed of 1000-2000 RPM, and the weight ratio between the polysaccharide-grafted N-isopropylacrylamide (NIPAAm) powder and the polypropylene (PP) powder is 1:5-1:10. Then, the polysaccharide grafted N-isopropylacrylamide (NIPAAm) powder and the polypropylene (PP) powder are processed into a polymer melt state by the plastic masterbatch processing equipment, and the blending and grafting of the polysaccharide-grafted N-isopropylacrylamide (NIPAAm) powder and the polypropylene (PP) powder (i.e. the polymer) are achieved in such a melt state, wherein a schematic view of the graft structure is shown in FIG. 6, to finally produce a plastic masterbatch of a polysaccharide-grafted N-isopropylacrylamide (NIPAAm) blended polymer, wherein an added amount of the grafting agent (i.e. N-isopropylacrylamide (NIPAAm)) is between 0.1-1% weight percentage relative to the polymer.

The main technical feature of the above-described production method for degrading cocoa shell into polysaccharide for grafting onto polymer is that cocoa shells, which are agricultural waste derived from a chocolate manufacturing process, are reused, mainly using anaerobic thermophilic lignin decomposing bacteria to have the cocoa shell degraded to form monosaccharides or polysaccharides, and then sorbitol and citric acid are added for high-temperature melting to polymerize into polysaccharide, and finally, N-isopropylacrylamide (NIPAAm) is added and covalently bonded, through grafting technology, to polymer to complete the production method for degrading cocoa shell into polysaccharide for grafting onto polymer according to the present invention. As such, a plastic masterbatch of a polysaccharide-grafted N-isopropylacrylamide (NIPAAm) blended polymer (such as polypropylene (PP)) is produced to thereby achieve greatly increasing the reuse rate of cocoa shell agricultural waste, and also, the polymer or masterbatch thereof produced according to the present invention can be soaked in sea water to indeed fulfill an effect of degradation, and avoiding polluting the marine ecology and environment.

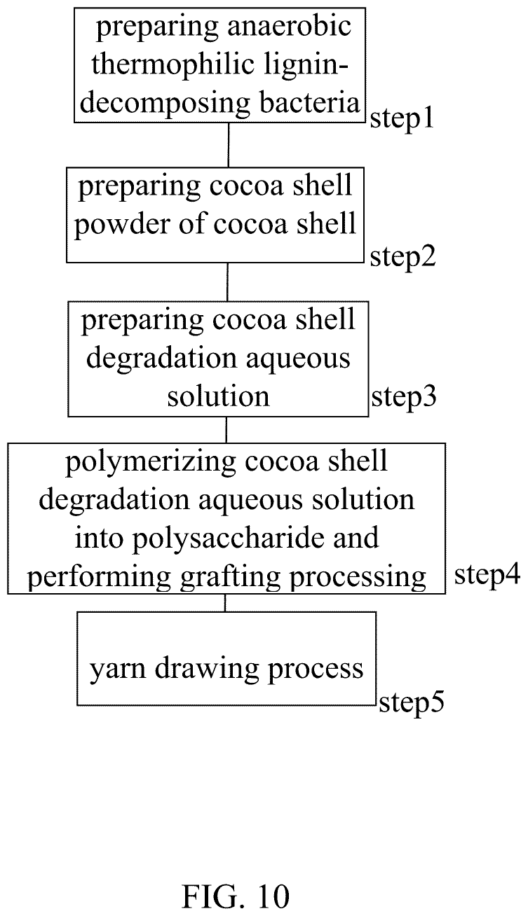

The present invention also discloses a production method for yarn of cocoa shell degraded into polysaccharide grafted onto polymer. Referring to FIG. 10, the production method includes: Steps 1-4 of the above-described production method for degrading cocoa shell into polysaccharide for grafting onto polymer, followed by Step 5, a yarn drawing process: using preset plastic masterbatch manufacturing equipment to make a polysaccharide grafted N-isopropylacrylamide (NIPAAm) blended polymer into a plastic masterbatch of polysaccharide-grafted N-isopropylacrylamide (NIPAAm) blended polymer, and then subjecting the plastic masterbatch to a yarn drawing operation to make a textile yarn of a polysaccharide grafted N-isopropylacrylamide (NIPAAm) blended polymer, so that the textile yarn can be woven into fabric, significantly increasing the reuse rate of cocoa shell agricultural waste, and also, the textile yarn or fabric made by the present invention can be soaked in seawater to indeed fulfill an effect of degradation, and avoiding polluting the marine ecology and environment.

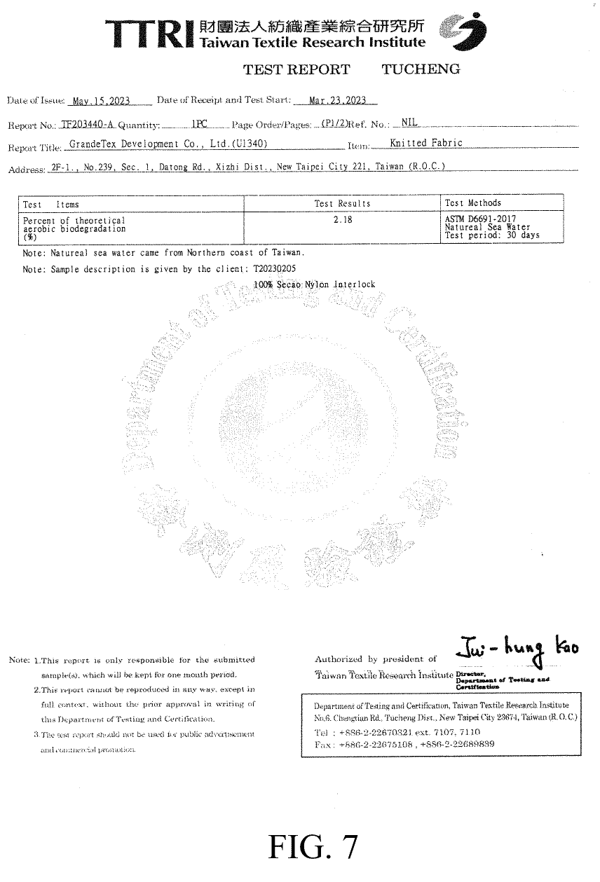

Referring to FIG. 7, a result of fabric degradation by seawater implemented on the fabric made of textile yarns that are made of polysaccharide grafted N-isopropylacrylamide (NIPAAm) blended polyester (PET) is shown. The result showed that the weight loss of the fabric was nearly more than 2%, and an effect of degradation is indeed achieved.

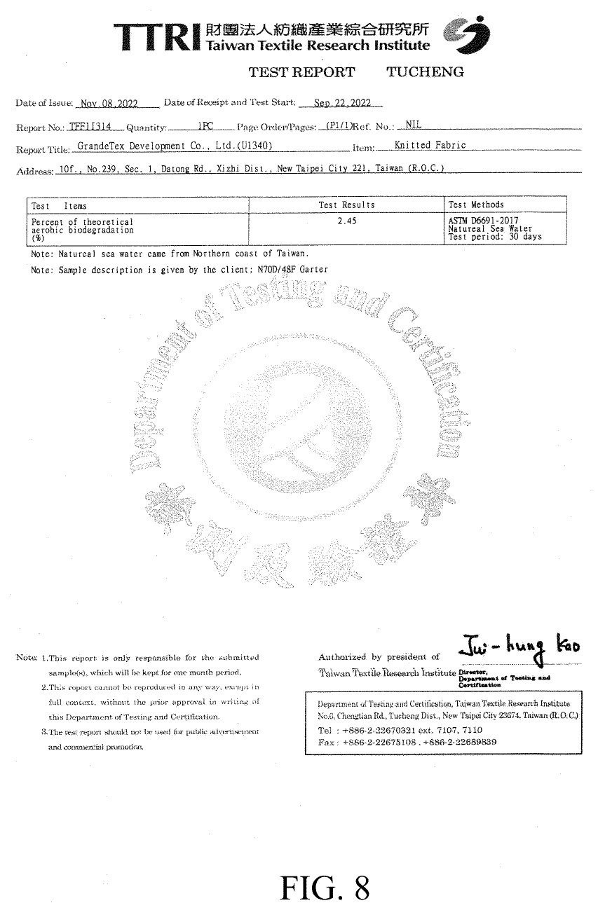

Referring to FIG. 8, a result of fabric degradation by seawater implemented on the fabric made of textile yarns that are made of polysaccharide grafted N-isopropylacrylamide (NIPAAm) blended nylon PA6 is shown. The result showed that the weight loss of the fabric was nearly more than 2%, and an effect of degradation is indeed achieved.

I claim:

1. A production method for degrading cocoa shell into polysaccharide for grafting onto polymer, comprising:
(1) preparing anaerobic thermophilic lignin-decomposing bacteria;
(2) preparing a cocoa shell powder of cocoa shell;
(3) preparing a cocoa shell degradation aqueous solution: adding the cocoa shell powder into a decomposing bacteria culture solution with the anaerobic thermophilic lignin-decomposing bacteria, wherein during a course of preparation, the pH value of the decomposing bacteria culture solution is set to be between 4-8 and after a preset time, the anaerobic thermophilic lignin-decomposing bacteria degrade the cocoa shell powder to obtain a cocoa shell degradation aqueous solution; and
(4) polymerizing the cocoa shell degradation aqueous solution into polysaccharide and performing grafting processing: subjecting the cocoa shell degradation aqueous solution to a centrifugation process to obtain a degraded saccharide liquid and setting a saccharide content of the saccharide liquid to be between 70%-80%, and adding sorbitol and citric acid to carry out high temperature melting for polymerization to obtain a polysaccharide powder; placing the polysaccharide powder and N-isopropylacrylamide (NIPAAm) in a reaction vessel filled with ethanol and added with an initiator to carry out a preset reaction process and carrying out a drying process after the reaction process is completed to obtain a polysaccharide-grafted N-isopropylacrylamide (NIPAAm) powder; and finally, subjecting the polysaccharide-grafted N-isopropylacrylamide (NIPAAm) powder to a blending process with a polymer to allow the polysaccharide-grafted N-isopropylacrylamide (NIPAAm) powder to covalently bond to the polymer to complete a polysaccharide-grafted N-isopropylacrylamide (NIPAAm) blended polymer.

2. The production method for degrading cocoa shell into polysaccharide for grafting onto polymer according to claim 1, wherein in Step 4, conditions set for the high temperature melting are that a set temperature is between 110-130 degrees Celsius, and a pressure is between 0.01-0.1 atm, such that the polysaccharide powder is obtained through polymerization under these conditions.

3. The production method for degrading cocoa shell into polysaccharide for grafting onto polymer according to claim 1, wherein in Step 4, the initiator comprises dibenzoyl peroxide, and a concentration of the initiator in ethanol is 1−8×10⁻³ M.

4. The production method for degrading cocoa shell into polysaccharide for grafting onto polymer according to claim 1, wherein in Step 4, the polymer comprises one of PET (polyethylene terephthalate), PA6 (polyamide (NYLON)), PP (polypropylene), PE (polyethylene), ABS (acrylonitrile-butadiene-styrene copolymer), PC (polycarbonate), PVDF (polyvinylidene fluoride), PS (polystyrene), PES (polyether styrene), PVC (polyvinyl chloride), PAN (polyacrylonitrile), and PA6 (nylon 6).

5. The production method for degrading cocoa shell into polysaccharide for grafting onto polymer according to claim 1, wherein in Sep 4, the blending process is performed with preset plastic masterbatch manufacturing equipment to blend, graft and bond the polysaccharide-grafted N-isopropylacrylamide (NIPAAm) powder and the polymer, and the plastic masterbatch processing equipment processes the polysaccharide-grafted N-isopropylacrylamide (NIPAAm) powder and the polymer into a polymer melt state so that the polysaccharide-grafted N-isopropylacrylamide (NIPAAm) powder and the polymer powder are bended and grafted in the melt state.

6. A production method for yarn of cocoa shell degraded into polysaccharide grafted onto polymer, comprising:
(1) a yarn drawing process: using preset plastic masterbatch manufacturing equipment to make a polysaccharide grafted N-isopropylacrylamide (NIPAAm) blended polymer into a plastic masterbatch of polysaccharide-grafted N-isopropylacrylamide (NIPAAm) blended polymer, and then subjecting the plastic masterbatch to a yarn drawing operation to make a textile yarn of the polysaccharide grafted N-isopropylacrylamide (NIPAAm) blended polymer.

7. The production method for yarn of cocoa shell degraded into polysaccharide grafted onto polymer according to claim 6, wherein in Step 4, conditions set for the high temperature melting are that a set temperature is between 110-130 degrees Celsius, and a pressure is between 0.01-0.1 atm, such that the polysaccharide powder is obtained through polymerization under these conditions.

8. The production method for yarn of cocoa shell degraded into polysaccharide grafted onto polymer according to claim 6, wherein in Step 4, the initiator comprises dibenzoyl peroxide, and a concentration of the initiator in ethanol is 1−8×10⁻³ M.

9. The production method for yarn of cocoa shell degraded into polysaccharide grafted onto polymer according to claim 6, wherein in Step 4, the polymer comprises one of PET (polyethylene terephthalate), PA6 (polyamide (NYLON)), PP (polypropylene), PE (polyethylene), ABS (acrylonitrile-butadiene-styrene copolymer), PC (polycarbonate), PVDF (polyvinylidene fluoride), PS (polystyrene), PES (polyether styrene), PVC (polyvinyl chloride), PAN (polyacrylonitrile), and PA6 (nylon 6).

10. The production method for yarn of cocoa shell degraded into polysaccharide grafted onto polymer according to claim 6, wherein in Sep 4, the blending process is performed with preset plastic masterbatch manufacturing equipment to blend, graft and bond the polysaccharide-grafted N-isopropylacrylamide (NIPAAm) powder and the polymer, and the plastic masterbatch processing equipment processes the polysaccharide-grafted N-isopropylacrylamide (NIPAAm) powder and the polymer into a polymer melt state so that the polysaccharide-grafted N-isopropylacrylamide (NIPAAm) powder and the polymer powder are bended and grafted in the melt state.
